(19) United States
(12) Patent Application Publication
(10) Pub. No.: US 2024/0335577 A1
(43) Pub. Date: Oct. 10, 2024

(54) STERILIZATION DEVICE

(71) Applicant: Enplas Corporation, Saitama (JP)

(72) Inventors: Naohiro ISHIKAWA, Saitama (JP); Toshinori OKADA, Saitama (JP); Kakeru NAKAMURA, Saitama (JP)

(73) Assignee: Enplas Corporation, Saitama (JP)

(21) Appl. No.: 18/574,339

(22) PCT Filed: May 31, 2022
(86) PCT No.: PCT/JP2022/022049

§ 371 Date: Dec. 27, 2023

(30) Foreign Application Priority Data
Jun. 30, 2021 (JP) 2021-109443

(51) Int. Cl. A61L 2/10; A61L 2/26; C02F 1/32

(52) U.S. Cl. CPC A61L 2/10; A61L 2/26; C02F 1/325; A61L 2202/11; A61L 2202/122; C02F 2201/3222; C02F 2303/04

(57) ABSTRACT

The present invention relates to a sterilization device comprises: a storage part that is for storing a fluid and has a substantially spherical shape; a supply port for supplying the fluid into the storage part; an outlet for extracting the fluid from the storage part; and a light source for radiating ultraviolet light. When the supply port and the outlet are projected on an imaginary plane orthogonal to the extending direction of the inner surface of a supply channel that is connected to the storage part in the supply port, the centroid of the supply port is spaced apart from the centroid of the outlet. The light source is disposed such that the optical axis thereof is positioned at 75° to 105° with respect to a straight line that connects the centroid the storage part and the centroid of the outlet.

### TECHNICAL FIELD

The present invention relates to a sterilization apparatus that sterilizes a fluid by irradiating the fluid with ultraviolet light.

### BACKGROUND ART

It is widely known that ultraviolet light can be used to sterilize liquids and other fluids. For example, Patent Literature 1 describes a fluid sterilization apparatus that emits ultraviolet light in the axial direction of the apparatus into a channel extending in the axial direction to sterilize fluid flowing in the channel.

Specifically, the fluid sterilization apparatus described in Patent Literature 1 includes a light source including a semiconductor light emitting element that emits ultraviolet light, and a housing including a channel through which a fluid to be sterilized flows in the axial direction. The light source is disposed at one end of the housing in the axial direction. The housing has a tapered structure in which the cross-sectional area of the channel gradually increases from the one end to the other end. The tapered structure has an inclination that matches the orientation angle of the semiconductor light emitting device. Further, a rectifying means for adjusting the flow of the fluid is provided at the other end of the housing.

According to Patent Literature 1, as the housing has a tapered structure with an inclination that matches the orientation angle of the semiconductor light emitting device, the sterilizing effect can be increased because the fluid is evenly irradiated with ultraviolet light by allowing the ultraviolet light to reach locations far from the light source, and by irradiating the fluid whose flow has been adjusted by the rectifying means with the ultraviolet light.

### CITATION LIST

### Patent Literature

### Patent Literature 1

Japanese Patent Application Laid-Open No. 2019-98055

### SUMMARY OF INVENTION

### Technical Problem

The fluid sterilization apparatus described in Patent Literature 1 is not enough from the viewpoint of effectively irradiating a fluid with ultraviolet light.

An object of the present invention is to provide a sterilization apparatus capable of sterilizing a fluid by irradiating the fluid with ultraviolet light effectively.

### Solution to Problem

A sterilization apparatus according to an embodiment of the present invention is a sterilization apparatus configured to sterilize a fluid by irradiating the fluid with ultraviolet light, and the sterilization apparatus includes:
a storage part for storing the fluid, the storage part having a substantially spherical shape; a supply port for supplying the fluid into the storage part, the supply port opening into the storage part; an outlet port for taking out the fluid within the storage part, the outlet port opening into the storage part; and a light source for emitting the ultraviolet light into the storage part, in which
the storage part includes a first storage part that has a substantially hemispherical shape and is located on an upstream side in a flow direction of the fluid at the supply port, and a second storage part that has a substantially hemispherical shape and is located on a downstream side in the flow direction,
the supply port opens into the first storage part,
the outlet port opens into the second storage part,
when the supply port and the outlet port are projected onto a virtual plane, a center of gravity of the supply port and a center of gravity of the outlet port are separated from each other, the virtual plane being a virtual plane orthogonal to an extending direction of an inner surface of a supply channel connected to the storage part at the supply port, and
the light source is disposed so that an optical axis thereof is at an angle of 75° to 105° with respect to a straight line connecting a center of gravity of the storage part with the center of gravity of the outlet port.

### Advantageous Effects of Invention

The present invention can provide a sterilization apparatus that can effectively irradiate a fluid with ultraviolet light to sterilize the fluid.

### BRIEF DESCRIPTION OF DRAWINGS

FIG. 1 is a perspective view of a sterilization apparatus according to an embodiment;

FIG. 2 is a cross-sectional perspective view of the sterilization apparatus according to the embodiment;

FIG. 3 is a projection view illustrating the positional relationship between a supply port and an outlet port when the supply port, the outlet port, and a storage part are projected onto a virtual plane; and

FIG. 4 illustrates the flow of a fluid within the storage part of the sterilization apparatus according to the embodiment.

### DESCRIPTION OF EMBODIMENTS

Hereinafter, a sterilization apparatus according to an embodiment of the present invention will be described.

### Configuration of Sterilization Apparatus

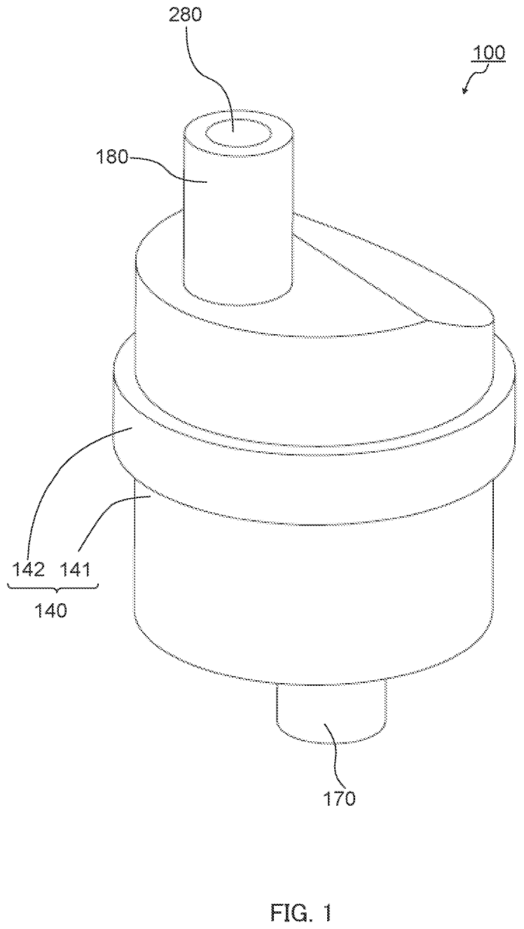

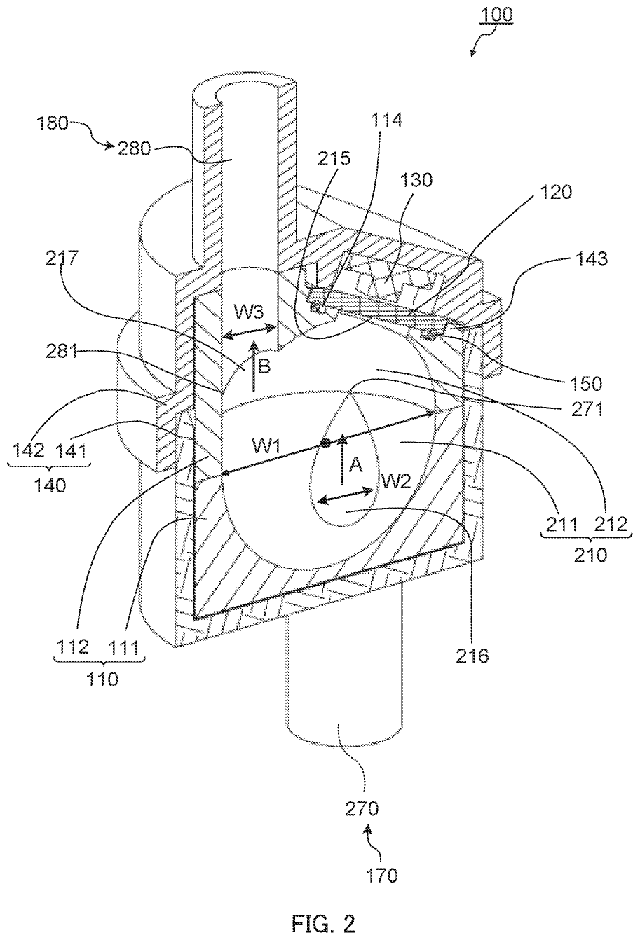

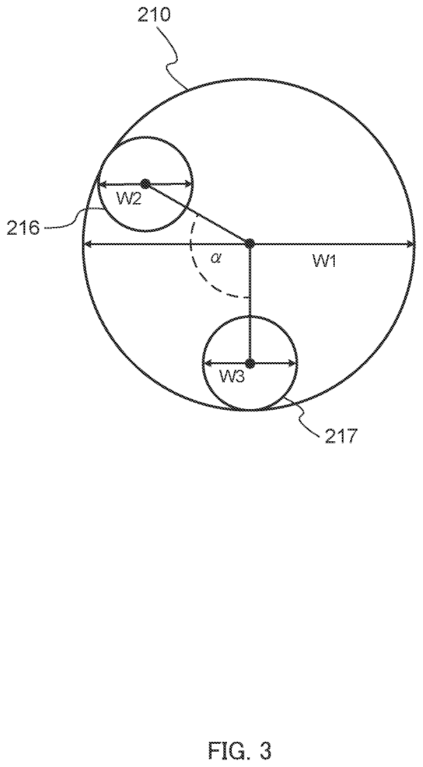

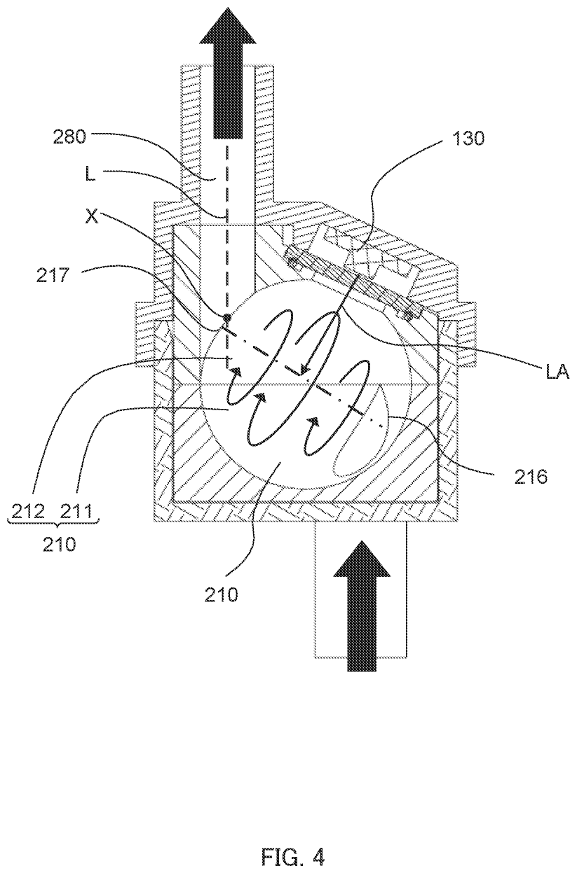

FIGS. 1 and 2 illustrate a configuration of sterilization apparatus 100 according to an embodiment of the present invention. FIG. 1 is a perspective view of sterilization apparatus 100. FIG. 2 is a cross-sectional perspective view of sterilization apparatus 100. FIG. 3 is a projection view illustrating the positional relationship between supply port 216 and outlet port 217 when supply port 216, outlet port 217, and storage part 210 are projected onto a virtual plane (described below). FIG. 4 illustrates the flow of a fluid within storage part 210.

As illustrated in FIGS. 1 and 2, sterilization apparatus 100 is configured to sterilize a fluid by irradiating the fluid with ultraviolet light. Sterilization apparatus 100 includes inner wall part 110, window 120, light source 130, outer wall part 140, and sealing member 150. In addition to the above configuration, sterilization apparatus 100 of the present embodiment further includes supply part 170 and outlet part 180.

Inner wall part 110 forms storage part 210, irradiation port 215, supply port 216, and outlet port 217. Storage part 210 is a substantially spherical space disposed inside inner wall part 110 and is for storing a fluid. Irradiation port 215 is a through hole that opens into storage part 210 and the outside and is configured to guide ultraviolet light from the outside (light source 130) into storage part 210. Supply port 216 is a through hole that opens into storage part 210 and the outside and is for supplying a fluid into storage part 210. Outlet port 217 is a through hole that opens into storage part 210 and the outside and is for taking out a fluid within storage part 210.

Inner diameter W1 of storage part 210 is not limited. Inner diameter W1 of storage part 210 is, for example, approximately 10 to 60 mm. Setting inner diameter W1 of storage part 210 to approximately 10 to 60 mm allows the fluid within inner wall part 110 to be satisfactorily sterilized even when only one UV-C LED is used as light source 130.

The ratio of the inner diameter of irradiation port 215 to inner diameter W1 of storage part 210 is preferably 20 to 50%. Increasing the inner diameter of irradiation port 215 allows a wide range of storage part 210 to be directly irradiated with ultraviolet light. On the other hand, reducing the inner diameter of irradiation port 215 allows the ratio of the ultraviolet reflective surface to the inner surface of storage part 210 to increase.

Inner wall part 110 may be formed of one member or may be formed of a plurality of members. In the present embodiment, inner wall part 110 is formed of two members, namely first inner wall part 111 and second inner wall part 112. In addition, inner wall part 110 forms storage part 210, irradiation port 215, supply port 216, and outlet port 217.

First inner wall part 111 forms supply port 216 and first storage part 211 that has a substantially hemispherical shape and is located on the upstream side in the flow direction (direction of arrow A illustrated in FIG. 2) of a fluid at supply port 216. Supply channel 270 is connected to supply port 216.

Second inner wall part 112 forms outlet port 217, irradiation port 215, and second storage part 212 that has a substantially hemispherical shape and is located on the downstream side in the flow direction (direction of arrow A illustrated in FIG. 2) of a fluid at supply port 216. Outlet channel 280 is connected to outlet port 217. Irradiation port 215 is covered by window 120, and the ultraviolet light emitted from light source 130 passes through irradiation port 215. In the present embodiment, annular groove 114 is disposed in the outer surface of second inner wall part 112.

In the above description, supply port 216 is in first inner wall part 111, and outlet port 217 is in second inner wall part 112; however, the configurations of supply port 216 and outlet port 217 are not limited thereto. Supply port 216 may be configured to be located on both first inner wall part 111 and second inner wall part 112, and outlet port 217 may be configured to be located on both second inner wall part 112 and first inner wall part 111. That is, supply port 216 may open into both first storage part 211 and second storage part 212, and outlet port 217 may open into both second storage part 212 and first storage part 211.

Annular groove 114 is a groove for positioning sealing member 150. Annular groove 114 may have any shape as long as sealing member 150 can be appropriately disposed within a range so as to not obstruct light from light source 130. In the present embodiment, annular groove 114 is disposed to surround irradiation port 215. Sealing member 150 can be deformed following the shape of annular groove 114; therefore, the shape of annular groove 114 may be the same as or different from the shape of sealing member 150. In the present embodiment, annular groove 114 has an elliptical shape in plan view, and the circular sealing member 150 is deformed and fitted therein.

Inner wall part 110 is formed of a member that is not deformed or damaged by the pressure of the flowing fluid. Examples of materials for inner wall part 110 include metals such as aluminum and resins such as polytetrafluoroethylene (PTFE). In addition, from the viewpoint of efficiently irradiating a fluid within inner wall part 110 with ultraviolet light, the inner surface of inner wall part 110 preferably includes an ultraviolet reflective surface having a reflectance of 80% or more of the ultraviolet light emitted from light source 130. Examples of materials for the ultraviolet reflective surface include aluminum and polytetrafluoroethylene (PTFE), which have high reflectance of ultraviolet light. In addition, the ultraviolet reflective surface may be formed by applying an ultraviolet reflective paint to the inner surface of inner wall part 110 or by forming an ultraviolet reflective film on the inner surface. In the present embodiment, the material of inner wall part 110 (both of first inner wall part 111 and second inner wall part 112) is PTFE.

Window 120 is disposed to cover irradiation port 215. Window 120 may have any shape as long as the window can transmit ultraviolet light emitted from light source 130 to storage part 210. Window 120 may have a shape of a flat plate, or a shape that matches the inner surface of storage part 210. In the present embodiment, window 120 has a shape of a flat plate. Window 120 may have any size as long as the window completely blocks irradiation port 215 and allows sealing member 150 to be appropriately disposed between inner wall part 110 and window 120.

Window 120 may be made of any material as long as window 120 can transmit ultraviolet light and has the necessary strength. From the viewpoint of improving sterilization performance, the material for window 120 is preferably a material that allows transmission of ultraviolet light having wavelength of 200 nm or more and 350 nm or less, and more preferably a material that allows transmission of ultraviolet light having a wavelength of 200 nm or more and 280 nm or less. Examples of materials for window 120 include quartz (SiO₂), sapphire (Al₂O₃), and amorphous fluorine resin.

Light source 130 irradiates a fluid in storage part 210 with ultraviolet light. Light source 130 may directly irradiate a fluid in storage part 210 with ultraviolet light, or may irradiate the fluid in storage part 210 with ultraviolet light through another member such as a window. In the present embodiment, light source 130 is fixed to outer wall part 140 and emits ultraviolet light into storage part 210 through window 120. More specifically, light source 130 is disposed on the second storage part 212 side in the present embodiment. Light source 130 may be of any type as long as light source 130 can emit ultraviolet light. Examples of light source 130 include light emitting diodes (LEDs), mercury lamps, metal halide lamps, xenon lamps, and laser diodes (LDs). In the present embodiment, light source 130 is a light emitting diode (LED). The wavelength of the ultraviolet light emitted by light source 130 is not limited. From the viewpoint of effective sterilization, the wavelength of the ultraviolet light emitted by light source 130 is preferably 200 nm or more and 350 nm or less, and more preferably 200 nm or more and 280 nm or less. That is, the ultraviolet light emitted from light source 130 are preferably ultraviolet C rays (UVC). Examples of commercially available light sources 130 include NCSU334A (Nichia Corporation), which is an ultraviolet light emitting diode with a peak wavelength of 280 nm. In addition, other examples of ultraviolet light emitting diodes having a peak wavelength of 280 nm include KLARAN (Asahi Kasei Corp.) and ZEU110BEAE (Stanley Electric co. Ltd.).

In the above description, light source 130 is disposed on the second storage part 212 side; however, the sterilization apparatus of the present invention is not limited thereto. Light source 130 may be disposed on the first storage part 211 side.

Light source 130 is preferably disposed so that optical axis LA thereof is at an angle of 75° to 105° with respect to the straight line connecting the center of gravity of storage part 210 with the center of gravity of outlet port 217 (more precisely, the opening of outlet port 217 to storage part 210). The angle is more preferably 80° to 100°, and even more preferably 85° to 95°. As illustrated in FIG. 4, optical axis LA is disposed at 90° with respect to the above straight line in the present embodiment. In addition, optical axis LA preferably intersects with the center of gravity of storage part 210. Disposing light source 130 in this manner allows efficient irradiation of a fluid with ultraviolet light.

The center of gravity of storage part 210 and the center of gravity of outlet port 217 in the present embodiment are as follows. As illustrated in FIG. 3, when (the opening of) outlet port 217 and storage part 210 are projected onto a virtual plane—the virtual plane is orthogonal to the extending direction (direction of arrow A in FIG. 2) of the inner surface of supply channel 270 connected to storage part 210 at supply port 216, the opening and storage part 210 are both circular. Therefore, the center of gravity of (the opening of) outlet port 217 coincides with the center of (the opening of) outlet port 217, and the center of gravity of storage part 210 coincides with the center of the storage part 210, in the present embodiment. In addition, as illustrated in FIG. 4, the center of gravity of outlet port 217 is intersection X between center line L of outlet channel 280 and the virtual surface of the sphere of storage part 210, in the present embodiment. In addition, optical axis LA is referred to as the central light of the three-dimensional light flux emitted from light source 130.

As illustrated in FIG. 4, in storage part 210, a fluid flowing from supply port 216 to outlet port 217 is more likely to circle around the straight line connecting the center of gravity of storage part 210 with the center of gravity of outlet port 217 as the central axis. Disposing light source 130 as described above allows irradiation with ultraviolet light at an angle of approximately 90° with respect to the circling axis, thereby achieving effective sterilization.

The angle of the circling axis changes depending on the amount of the fluid supplied into storage part 210 or the like. However, disposing light source 130 at an angle within the above range sets optical axis LA at approximately 90° with respect to the circling axis, thereby effectively irradiating the fluid with ultraviolet light.

Outer wall part 140 covers inner wall part 110 and window 120 and presses inner wall part 110 and window 120. In the present embodiment, outer wall part 140 covers light source 130 in addition to inner wall part 110 and window 120. In the present embodiment, outer wall part 140 includes first outer wall part 141 and second outer wall part 142.

First outer wall part 141 covers first inner wall part 111 from the upstream side in the flow direction of a fluid (herein also referred to as “fluid flow direction”). In the present embodiment, first outer wall part 141 is disposed to cover first inner wall part 111 and a portion of second inner wall part 112 on the first inner wall part 111 side.

Second outer wall part 142 covers second inner wall part 112 from the downstream side in the fluid flow direction. In the present embodiment, second outer wall part 142 is disposed to cover a portion of second inner wall part 112 on the downstream side in the fluid flow direction. In addition, positioning step part 143 for positioning window 120 is disposed at second outer wall part 142. Positioning step part 143 is disposed to surround irradiation port 215. In addition, light source 130 is disposed to face window 120 positioned by positioning step part 143.

First inner wall part 111 can be joined with second inner wall part 112 by joining first outer wall part 141 with second outer wall part 142. In the present embodiment, in the fluid flow direction, the joint portion between first outer wall part 141 and second outer wall part 142 does not coincide with the joint portion between first inner wall part 111 and second inner wall part 112. More specifically, the joint portion between first outer wall part 141 and second outer wall part 142 is located downstream of the joint portion between first inner wall part 111 and second inner wall part 112 in the fluid flow direction. Any method may be used for joining first outer wall part 141 with second outer wall part 142. In the present embodiment, first outer wall part 141 and second outer wall part 142 are joined by fitting.

Sealing member 150 is an elastic member disposed between inner wall part 110 and window 120, and seals a space between inner wall part 110 and window 120. Sealing member 150 is disposed between inner wall part 110 and window 120 so as to surround irradiation port 215. Sealing member 150 may have any configuration as long as inner wall part 150 can appropriately seal the space between inner wall part 110 and window 120. Sealing member 150 is, for example, an O-ring or packing. In the present embodiment, sealing member 150 is an O-ring. In the present embodiment, sealing member 150 (O-ring) is disposed in annular groove 114 of second inner wall part 112. In the present embodiment, second inner wall part 112 in which sealing member 150 and window 120 are disposed in this order is housed in second outer wall part 142 to which light source 130 is fixed.

Supply part 170 supplies a fluid into storage part 210 located in inner wall part 110. Supply part 170 includes supply channel 270. One end of supply channel 270 is connected to supply port 216 of inner wall part 110, and the other end of supply channel 270 is connected to a fluid supply apparatus which is not illustrated. Supply channel 270 is preferably disposed so as to be able to smoothly supply a fluid into storage part 210 along the wall of storage part 210. In the present embodiment, a portion of the inner surface of supply channel 270 is disposed to be smoothly continuous with the inner surface of storage part 210 in first connection part 271 in such a way that the portion coincides with a tangent to the inner surface of storage part 210 in first connection part 271. Here, first connection part 271 is located between the inner surface of supply channel 270 and the inner surface of storage part 210 in a cross section that is along the flow direction (direction of arrow A illustrated in FIG. 2) of a fluid at supply port 216 and includes the center of gravity of storage part 210.

Outlet part 180 takes out the sterilized fluid within storage part 210 located in inner wall part 110. Outlet part 180 forms outlet channel 280. One end of outlet channel 280 is connected to outlet port 217 of inner wall part 110, and the other end of outlet channel 280 is connected to a fluid outlet apparatus which is not illustrated. Outlet part 180 is preferably disposed in storage part 210 (second storage part 212) at a position where the ultraviolet light emitted from light source 130 does not directly reach. Outlet channel 280 is preferably disposed so that the fluid can be smoothly taken out from storage part 210 along the wall of storage part 210. In the present embodiment, a portion of the inner surface of outlet channel 280 is disposed to be smoothly continuous with the inner surface of storage part 210 in second connection part 281 in such a way that the portion coincides with a tangent to the inner surface of storage part 210 in second connection part 281. Here, second connection part 281 is located between the inner surface of outlet channel 280 and the inner surface of storage part 210 in a cross section that is along the flow direction (direction of arrow B illustrated in FIG. 2) of a fluid at outlet port 217 and includes the center of gravity of storage part 210. In the present embodiment, supply channel 270 and outlet channel 280 are parallel to each other.

No step is formed at first connection part 271 between the inner surface of supply channel 270 and the inner surface of storage part 210, and at second connection part 281 between the inner surface of outlet channel 280 and the inner surface of storage part 210. It is thus possible to create a flow of a fluid along the wall surface of the spherical storage part 210, and also to take out the fluid after the fluid is retained in storage part 210 while being rotated in a certain direction. Such a configuration allows the fluid to be uniformly irradiated with ultraviolet light, thereby satisfactorily sterilizing the fluid.

Inner diameter W2 of supply port 216 (supply channel 270) and inner diameter W3 of outlet port 217 (outlet channel 280) may have any value. However, inner diameter W2 and inner diameter W3 are preferably within a range of 25 to 40% of inner diameter W1 of storage part 210 from the viewpoint of reducing fluid pressure loss while maintaining sterilization performance. Increasing inner diameter W2 of supply port 216 and inner diameter W3 of outlet port 217 can reduce the pressure loss of the fluid in sterilization apparatus 100. On the other hand, reducing inner diameter W2 of supply port 216 and inner diameter W3 of outlet port 217 can lengthen the duration in which a fluid supplied from supply port 216 remains in storage part 210, thereby improving the sterilization performance. More specifically, ratios of inner diameter W2 of supply port 216 (supply channel 270) and inner diameter W3 of outlet port 217 (outlet channel 280) to inner diameter W1 of storage part 210 may be 10% or more.

As illustrated in FIG. 3, when supply port 216, outlet port 217, and storage part 210 are projected onto a virtual plane defined as follows, supply port 216 and outlet port 217 are disposed in such a way that the center of gravity of supply port 216 and the center of gravity of outlet port 217 are separated from each other. The virtual plane is a virtual plane orthogonal to the extending direction (direction of arrow A in FIG. 2) of the inner surface of supply channel 270 connected to storage part 210 at supply port 216. In the present embodiment, supply port 216 and outlet port 217 are disposed in such a way that supply port 216 and outlet port 217 are separated from each other when those elements are projected as described above. When supply port 216 and outlet port 217 are disposed in this manner, the fluid supplied into storage part 210 from supply port 216 does not flow straight to outlet port 217, but instead circles within storage part 210 many times before reaching outlet port 217, as illustrated in FIG. 4. Therefore, the fluid reaches outlet port 217 after being irradiated with a satisfactory amount of ultraviolet light and satisfactorily sterilized. In the present embodiment, window 120 (light source 130) is disposed so as not to overlap supply port 216 and outlet port 217 when those elements are projected as described above. In the present embodiment, supply port 216, outlet port 217, and storage part 210 are all circular when those elements are projected onto the virtual plane as illustrated in FIG. 4. Therefore, the center of gravity of supply port 216 coincides with the center of supply port 216, the center of gravity of outlet port 217 coincides with the center of outlet port 217, and the center of gravity of storage part 210 coincides with the center of storage part 210.

In addition, in the present embodiment, when supply port 216, outlet port 217, and storage part 210 are projected onto the virtual plane as illustrated in FIG. 3, angle a between the straight line connecting the center of gravity of supply port 216 with the center of gravity of storage part 210 and the straight line connecting the center of gravity of outlet port 217 with the center of gravity of storage part 210 is preferably within the range of 75° to 165°, more preferably within the range of 120° to 150°. Setting angle a within the above range can further reduce the pressure loss of the fluid while maintaining satisfactory sterilization performance. Herein, an angle between two straight lines is referred to as the smaller of the two angles between the two straight lines.

### Method for Using Sterilization Apparatus

Hereinafter, a method for using sterilization apparatus 100 according to the present embodiment will be described.

While ultraviolet light is emitted from light source 130, a fluid to be sterilized (for example, water) is introduced into storage part 210 through supply port 216, and a fluid within storage part 210 is taken out through outlet port 217. At this time, the fluid may be moved by pressurizing from the supply port 216 (supply channel 270) side, or by depressurizing from the outlet port 217 (outlet channel 280) side. As described above, storage part 210 has a substantially spherical shape, and supply port 216 and outlet port 217 are disposed to satisfy predetermined conditions in sterilization apparatus 100 according to the present embodiment; therefore, a fluid to be sterilized circles within storage part 210. Further, as described above, light source 130 is disposed at an angle of 75° to 105° with respect to the straight line connecting the center of gravity of storage part 210 with the center of gravity of (the opening of) outlet port 217. As a result, the fluid is taken out from outlet port 217 in a satisfactorily sterilized state.

### Effects

By disposing light source 130 as described above, the fluid can be effectively sterilized in sterilization apparatus 100 according to the present embodiment.

This application is entitled to and claims the benefit of Japanese Patent Application No. 2021-109443 filed on Jun. 30, 2021, the disclosure of which including the specification and drawings is incorporated herein by reference in its entirety.

### INDUSTRIAL APPLICABILITY

The sterilization apparatus according to the present embodiment is particularly advantageous, for example, for sterilizing fluids, such as purified water, agricultural water, food washing water, various washing waters, bath water, and pool water.

### REFERENCE SIGNS LIST

100 Sterilization apparatus
110 Inner wall part
111 First inner wall part
112 Second inner wall part
114 Annular groove
120 Window
130 Light source
140 Outer wall part
141 First outer wall part
142 Second outer wall part
143 Positioning step part
150 Scaling member
170 Supply part
180 Outlet part
210 Storage part
211 First storage part
212 Second storage part
215 Irradiation port
216 Supply port
217 Outlet port
270 Supply channel
271 First connection part
280 Outlet channel
281 Second connection part

1. A sterilization apparatus configured to sterilize a fluid by irradiating the fluid with ultraviolet light, the sterilization apparatus comprising:
a storage part for storing the fluid, the storage part having a substantially spherical shape;
a supply port for supplying the fluid into the storage part, the supply port opening into the storage part;
an outlet port for taking out the fluid within the storage part, the outlet port opening into the storage part; and
a light source for emitting the ultraviolet light into the storage part,
wherein
the storage part includes a first storage part that has a substantially hemispherical shape and is located on an upstream side in a flow direction of the fluid at the supply port, and a second storage part that has a substantially hemispherical shape and is located on a downstream side in the flow direction,
the supply port opens into the first storage part,
the outlet port opens into the second storage part,
when the supply port and the outlet port are projected onto a virtual plane, a center of gravity of the supply port and a center of gravity of the outlet port are separated from each other, the virtual plane being a virtual plane orthogonal to an extending direction of an inner surface of a supply channel connected to the storage part at the supply port, and
the light source is disposed so that an optical axis thereof is at an angle of 75° to 105° with respect to a straight line connecting a center of gravity of the storage part with the center of gravity of the outlet port.

2. The sterilization apparatus according to claim 1, wherein the center of gravity of the storage part is separated from a straight line connecting the center of gravity of the supply port with the center of gravity of the outlet port.

3. The sterilization apparatus according to claim 2, wherein the center of gravity of the storage part is not located between the supply port and the outlet port.

4. The sterilization apparatus according to claim 1, wherein the outlet port is disposed at a position where the ultraviolet light emitted from the light source does not directly reach.

5. The sterilization apparatus according to claim 1, the light source is disposed on a side of the second storage part.

6. The sterilization apparatus according to claim 1, wherein:
a wall forming the storage part includes a window allowing transmission of the ultraviolet light; and
the light source emits the ultraviolet light into the storage part through the window.
